(19) United States
(12) Patent Application Publication
(10) Pub. No.: US 2024/0112792 A1
(43) Pub. Date: Apr. 4, 2024

(54) MEDICAL CARE SUBJECT INFORMATION MANAGEMENT SYSTEM AND MEDICAL CARE SUBJECT INFORMATION MANAGEMENT PROGRAM

(71) Applicant: TERUMO KABUSHIKI KAISHA, Tokyo (JP)

(72) Inventors: Kei HONDA, Kanagawa (JP); Ryotaro YABE, Kanagawa (JP)

(73) Assignee: TERUMO KABUSHIKI KAISHA, Tokyo (JP)

(21) Appl. No.: 18/474,488

(22) Filed: Sep. 26, 2023

(30) Foreign Application Priority Data
Sep. 30, 2022 (JP) 2022-157450

(51) Int. Cl. G16H 40/20; G16H 10/60; G16H 30/20; G16H 40/63

(52) U.S. Cl. CPC G16H 40/20; G16H 10/60; G16H 30/20; G16H 40/63

(57) ABSTRACT

The present disclosure improves handling of information related to a medical care without changing a flow of daily work already established by a health care worker. A medical care subject information management system is communicably connected to a reading device configured to read, from a measurement device configured to measure biological information on a medical care subject, the biological information measured by the measurement device, or includes such a reading device. The medical care subject information management system includes a control unit configured to cause a display unit to display an input screen for inputting information related to progress of a medical care of the medical care subject immediately or in response to a predetermined operation performed when the reading device reads the biological information on the medical care subject.

### CROSS-REFERENCES TO RELATED APPLICATIONS

This application claims priority to Japanese Application No. 2022-157450 filed on Sep. 30, 2022, the entire content of which is incorporated herein by reference.

### TECHNOLOGICAL FIELD

The present disclosure generally relates to a medical care subject information management system and a medical care subject information management program.

### BACKGROUND DISCUSSION

Japanese Patent Application Publication No. 2022-008980 A and Japanese Patent Application Publication No. 2016-130983 A describe techniques related to acquisition and display of medical care information.

However, techniques in the related art related to the acquisition and display of medical care information have room for improvement from a viewpoint of consistency with a flow of daily work already established by a health care worker.

### SUMMARY

The present disclosure improves handling of information related to a medical care without changing a flow of daily work already established by a health care worker.

(1) According to the present disclosure, a medical care subject information management system is communicably connected to a reading device configured to read, from a measurement device configured to measure biological information on a medical care subject, the biological information measured by the measurement device, or includes the reading device.

The medical care subject information management system includes a control unit configured to cause a display unit to display an input screen for inputting information related to progress of a medical care of the medical care subject immediately or in response to a predetermined operation performed when the reading device reads the biological information on the medical care subject.

(2) In the medical care subject information management system according to (1), the control unit may cause the display unit to display, as the input screen, a screen for inputting quantitative information related to the progress of the medical care of the medical care subject.

(3) In the medical care subject information management system (1) or (2), the control unit may cause the display unit to display, as the input screen, a screen on which an option can be selected from among a plurality of options related to the progress of the medical care.

(4) In the medical care subject information management system according to any one of (1) to (3), the control unit may cause the display unit to display, as the input screen, a screen including a progress image indicating a current progress status of the medical care of the medical care subject.

(5) In the medical care subject information management system according to any one of (1) to (4), in response to an input of the information related to the progress of the medical care of the medical care subject via the input screen, the control unit may cause the display unit to display, for a stage related to the progress of the medical care, a progress screen showing time information which is information related to a time required for the medical care of the medical care subject at this stage.

(6) In the medical care subject information management system according to (5), the control unit may cause the display unit to display, as the progress screen for the stage related to the progress of the medical care, a screen showing, as the time information, information on the number of times of rehabilitation performed on the medical care subject in the medical care at this stage.

(7) According to the present disclosure, a medical care subject information management system includes:

a control unit configured to cause a display unit to display an input screen for inputting information related to progress of a medical care of a medical care subject, the input screen including a progress image indicating a current progress status of the medical care of the medical care subject.

(8) In the medical care subject information management system according to (7), the control unit may cause the display unit to display, as the input screen, a screen further including, in addition to the progress image, a goal image which is an image indicating a goal corresponding to a progress status of the medical care of the medical care subject.

(9) In the medical care subject information management system according to (8), the control unit may cause the display unit to display, as the input screen, a screen including, as the goal image, an image indicating at least one of a most recent achievement goal according to the progress of the medical care of the medical care subject, a final achievement goal, and an achievement goal at each stage related to the progress of the medical care.

(10) In the medical care subject information management system according to any one of (7) to (9), the control unit may cause the display unit to display the input screen for inputting the information related to the progress of the medical care of the medical care subject, and the input screen includes display contents according to an occupation of a user who has logged in the medical care subject information management system.

(11) In the medical care subject information management system according to (10), when a user of a first occupation performs an input operation as an input person on the input screen for inputting the information related to the progress of the medical care of the medical care subject, the control unit may determine display contents on the input screen for a user of a second occupation based on an input result of the user of the first occupation, and may cause the display unit to display the input screen including the determined display contents for the user of the second occupation.

(12) According to the present disclosure, a medical care subject information management program stored on a non-transitory computer-readable medium that causes a computer that is communicably connected to a reading device configured to read, from a measurement device configured to measure biological information on a medical care subject, the biological information measured by the measurement device, or that includes the reading device to execute a process of causing a display unit to display an input screen for inputting information related to progress of a medical care of the medical care subject immediately or in response to a predetermined operation performed when the reading device reads the biological information on the medical care subject.

According to an embodiment of the present disclosure, it is possible to improve handling of information related to a medical care without changing a flow of daily work already established by a health care worker.

### BRIEF DESCRIPTION OF THE DRAWINGS

FIG. 1 is a diagram illustrating a configuration example of a medical system.

FIG. 2 is a flowchart illustrating an example of operations of a tablet terminal in FIG. 1.

FIG. 3 is a diagram illustrating an example of an image displayed on the tablet terminal in FIG. 1.

FIG. 4A is a diagram illustrating an example of an image displayed on the tablet terminal in FIG. 1.

FIG. 4B is a diagram illustrating an example of an image displayed on the tablet terminal in FIG. 1.

FIG. 4C is a diagram illustrating an example of an image displayed on the tablet terminal in FIG. 1.

FIG. 4D is a diagram illustrating an example of an image displayed on the tablet terminal in FIG. 1.

FIG. 5 is a diagram illustrating an example of a progress screen displayed on the tablet terminal in FIG. 1.

### DETAILED DESCRIPTION

Set forth below with reference to the accompanying drawings is a detailed description of embodiments of a medical care subject information management system and a medical care subject information management program. In the drawings, portions having the same configuration or function are denoted by the same reference signs. In the description of the present embodiment, the redundant description of the same portions may be omitted or simplified as appropriate.

Medical System

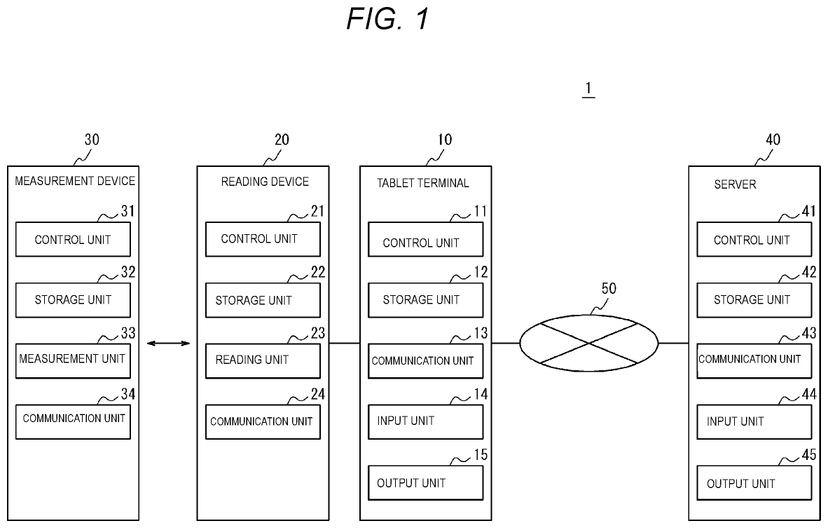

FIG. 1 is a diagram illustrating a configuration example of a medical system 1. As illustrated in FIG. 1, the medical system 1 includes a tablet terminal 10, a reading device 20, a measurement device 30, a server 40, and a network 50. The tablet terminal 10 and the server 40 are communicably connected to the network 50 including, for example, via the Internet and a mobile communication network. Hereinafter, an example will be described in which the number of the tablet terminal 10, the reading device 20, the measurement device 30, and the server 40 in the medical system 1 is one. Alternatively, the number of at least one of the tablet terminal 10, the reading device 20, the measurement device 30, and the server 40 may be two or more. A user of the tablet terminal 10, the reading device 20, and the measurement device 30 may be a medical care subject himself/herself, or may be a health care worker such as a doctor or a nurse.

The tablet terminal 10 of the medical care subject information management system according to the present embodiment can be an information processing device that acquires biological information on a medical care subject measured by the measurement device 30 from the reading device 20 and that displays an input screen of information (progress information) related to progress of a medical care of the medical care subject. The tablet terminal 10 transmits the acquired biological information, measurement date and time, and the like to the server 40 via the network 50, and receives, from the server 40, the progress information and basic data of the medical care subject, for example, such as a name, a gender, an age, a patient identifier (ID) for identifying a patient in a hospital, and a date of hospitalization. In the present embodiment, such a medical care subject information management system can be implemented by the tablet terminal 10. Alternatively, the medical care subject information management system may be implemented by any information processing device such as a smartphone or a personal computer (PC), or may be implemented by a unique device including necessary components.

In the present embodiment, the tablet terminal 10 is communicably connected to the reading device 20 via a universal serial bus (USB) cable. Alternatively, any communication method may be used between the tablet terminal 10 and the reading device 20. For example, the tablet terminal 10 may be communicably connected to the reading device 20 by a communication method such as a wired local area network (LAN), a wireless LAN, or Bluetooth®.

The reading device 20 is a device that reads, from the measurement device 30, biological information on a medical care subject measured by the measurement device 30. In the present embodiment, the reading device 20 reads the biological information from the measurement device 30 by near field communication (NFC). Alternatively, any communication method may be used between the reading device 20 and the measurement device 30. For example, the reading device 20 may read the biological information from the measurement device 30 by any communication method such as Bluetooth or a wireless LAN. When the reading device 20 reads the biological information from the measurement device 30, the reading device 20 transmits the biological information to the tablet terminal 10.

The measurement device 30 is a device that measures biological information on a medical care subject. Examples of such biological information include, and are not limited to, for example, a body temperature, a blood pressure (systolic blood pressure, diastolic blood pressure, and the like), a blood glucose level, oxygen saturation (SpO₂), a pulse, a respiration rate, a body weight, a fat weight, a lean body weight, a body water content, an intracellular fluid volume, an extracellular fluid volume, a cardiac output, an activity amount, and a basal body temperature (i.e., when the body is at rest). As described later, the measurement device 30 includes a communication unit 34 such as NFC that causes the reading device 20 to read the measured biological information.

The server 40 is an information processing device that manages an electronic medical record for each medical care subject. The electronic medical record records, for example, the biological information measured by the measurement device 30 and the progress information on the medical care subject are input into the tablet terminal 10. In the present embodiment, the server 40 can be implemented by a workstation (WS), but the server 40 may be implemented by any information processing device such as a personal computer (PC).

In the medical system 1, when the reading device 20 reads the biological information on the medical care subject from the measurement device 30, the tablet terminal 10 causes a display unit to display an input screen for inputting the progress information on the medical care subject immediately or in response to a predetermined operation being performed. As described above, since the tablet terminal 10 causes the display unit to display the input screen for inputting the progress information through a series of operations triggered by reading of the biological information using NFC or the like, the user can perform reading of the measured biological information and input of the progress information through a continuous operation. Therefore, according to the tablet terminal 10, it is possible to improve handling of information related to a medical care without changing a flow of daily work already established by a health care worker.

The biological information on the medical care subject read by the reading device 20 from the measurement device 30 may be transmitted not only from the tablet terminal 10 to the server 40 via the network 50, but also from the server 40 to the tablet terminal 10 via the network 50 after being first transmitted from the reading device 20 to the server 40 via the network 50. Further, instead of a configuration in which the reading device 20, the tablet terminal 10, and the server 40 are connected in series, the tablet terminal 10 and the server 40 may be connected in parallel to the reading device 20, and the biological information on the medical care subject read by the reading device 20 may be transmitted to both the tablet terminal 10 and the server 40.

When the tablet terminal 10 receives the biological information on the medical care subject measured by the measurement device 30, the tablet terminal 10 may cause the display unit to display the input screen for inputting the progress information on the medical care subject immediately or in response to the predetermined operation being performed, and at that time, the biological information measured by the measurement device 30 may or may not be displayed. When the biological information measured by the measurement device 30 is displayed, the tablet terminal 10 may display all pieces of the biological information obtained by the measurement device 30, may display a part of the obtained biological information, or may display information equivalent to the biological information by replacing the information with a sentence, a diagram, or the like.

Tablet Terminal

As illustrated in FIG. 1, the tablet terminal 10 includes a control unit 11, a storage unit 12, a communication unit 13, an input unit 14, and an output unit 15.

The control unit 11 includes at least one processor. The processor can include a general-purpose processor such as a central processing unit (CPU) or a specialized processor specialized for specific processing. The control unit 11 may include an application specific integrated circuit (ASIC), a digital signal processor (DSP), a programmable logic device (PLD), a field-programmable gate array (FPGA), or any combination of the ASIC, the DSP, the PLD and the FPGA. The control unit 11 may include a memory incorporated in the processor or a memory independent of the processor. The control unit 11 executes processing related to an operation of the tablet terminal 10 while controlling the units of the tablet terminal 10.

The storage unit 12 includes at least one semiconductor memory, at least one magnetic memory, at least one optical memory, or any combination of the at least one semiconductor memory, the at least one magnetic memory, and the at least one optical memory. The semiconductor memory can be, for example, a random access memory (RAM) or a read only memory (ROM). The RAM can be, for example, a static random access memory (SRAM) or a dynamic random access memory (DRAM). The ROM can be, for example, an electrically erasable programmable read only memory (EEPROM). The storage unit 12 can function as, for example, a main storage device, an auxiliary storage device, or a cache memory. The storage unit 12 can store data used for the operation of the tablet terminal 10 and data obtained by the operation of the tablet terminal 10. For example, the storage unit 12 may store the biological information on the medical care subject measured by the measurement device 30, the progress information on the medical care subject, and the like.

The communication unit 13 includes at least one communication interface. The communication unit 13 may include a communication interface for communicating with the reading device 20 and a communication interface for communicating with the server 40. The communication interface is an interface compatible with a mobile communication standard, for example, such as a USB, a wired LAN, a wireless LAN, Bluetooth, a long term evolution (LTE), a 4th generation (4G) standard or a 5th generation (5G) standard. The communication unit 13 receives the data used for the operation of the tablet terminal 10, and transmits the data obtained by the operation of the tablet terminal 10.

The input unit 14 includes at least one input interface. In the present embodiment, the input interface is a touch screen provided integrally with a display unit (display) of the output unit 15. Alternatively, any input interface may be used. For example, the input unit 14 may be a physical key, a static capacitance key, or a pointing device. The input unit 14 receives a user operation related to the operation of the tablet terminal 10. The input unit 14 may be connected to the tablet terminal 10 as an external input device instead of being provided in the tablet terminal 10.

The output unit 15 includes at least one output interface. The output interface can be, for example, a display (display unit). The display can be, for example, a liquid crystal display (LCD) or an organic electro luminescence (EL) display. The output unit 15 outputs the data obtained by the operation of the tablet terminal 10. The output unit 15 may be connected to the tablet terminal 10 as an external output device instead of being provided in the tablet terminal 10.

Functions of the tablet terminal 10 may be implemented by causing a processor serving as the control unit 11 to execute a program (medical care subject information management program) according to the present embodiment. The program causes a computer to function as the tablet terminal 10 by causing the computer to execute the operation of the tablet terminal 10. That is, the computer functions as the tablet terminal 10 by executing the operation of the tablet terminal 10 according to the program.

The program may be stored in a non-transitory computer-readable medium in advance. The non-transitory computer-readable medium can be, for example, a flash memory, a magnetic recording device, an optical disc, a magneto-optical recording medium, or a ROM. Distribution of the program is executed by, for example, selling, transferring, or lending a portable medium such as a secure digital (SD) card, a digital visual disc (DVD), or a compact disc read only memory (CD-ROM) that stores the program. The program may be distributed by storing the program in a storage of a cloud in advance and transferring the program from the cloud to another computer. The program may be provided as a program product.

For example, the computer temporarily stores, in the main storage device, the program stored in the portable medium or the program transferred from the cloud. The computer reads, by the processor, the program stored in the main storage device, and executes, by the processor, a process according to the read program. The computer may read the program directly from the portable medium and execute the process according to the program. Each time the program is transferred from the cloud to the computer, the computer may sequentially execute a process according to the received program. The process may be executed by a so-called application service provider (ASP) type service in which a function is implemented only by an execution instruction and acquisition of a result without transferring the program from the cloud to the computer. The program includes information provided for a process by an electronic computer and conforming to the program. For example, data that is not a direct command to the computer but has a property of defining a process of the computer corresponds to the “information conforming to the program”.

A part or all of the functions of the tablet terminal 10 may be implemented by a programmable circuit or a dedicated circuit as the control unit 11. That is, a part or all of the functions of the tablet terminal 10 may be implemented by hardware.

Reading Device

As illustrated in FIG. 1, the reading device 20 includes a control unit 21, a storage unit 22, a reading unit 23, and a communication unit 24.

The control unit 21 includes at least one processor. Any type and any configuration of the processor may be used as in a case of the processor of the control unit 11 of the tablet terminal 10. The control unit 21 executes a process related to an operation of the reading device 20 while controlling the units of the reading device 20.

The storage unit 22 includes at least one memory. Any type and any configuration of the memory may be used as in a case of the at least one memory of the storage unit 12 of the tablet terminal 10. The storage unit 22 functions as, for example, a main storage device, an auxiliary storage device, or a cache memory. The storage unit 22 may store data used for the operation of the reading device 20 and data obtained by the operation of the reading device 20. For example, the storage unit 22 may store the biological information or the like on the medical care subject measured by the measurement device 30.

The reading unit 23 includes at least one interface for reading the biological information on the medical care subject from the measurement device 30. For example, the reading unit 23 may include an interface of any communication method such as near field communication (NFC), Bluetooth, or a wireless LAN.

The communication unit 24 includes at least one communication interface. The communication unit 24 may include a communication interface for communicating with the tablet terminal 10. The communication interface may be any interface such as a USB cable, a wired LAN, a wireless LAN, or Bluetooth. The communication unit 24 may transmit, to the tablet terminal 10, the biological information on the medical care subject read from the measurement device 30 by the reading unit 23.

The reading device 20 may be provided in the tablet terminal 10, and the reading device 20 provided in the tablet terminal 10 may read the biological information on the medical care subject measured by the measurement device 30 by communicating with the measurement device 30 by wireless communication using a wireless method such as Bluetooth or NFC.

Measurement Device

As illustrated in FIG. 1, the measurement device 30 includes a control unit 31, a storage unit 32, a measurement unit 33, and the communication unit 34.

The control unit 31 includes at least one processor. Any type and any configuration of the processor may be used as in the case of the control unit 11 of the tablet terminal 10. The control unit 31 executes a process related to an operation of the measurement device 30 while controlling the units of the measurement device 30.

The storage unit 32 includes at least one memory. Any type and any configuration of the memory may be used as in the case of the storage unit 12 of the tablet terminal 10. The storage unit 32 functions as, for example, a main storage device, an auxiliary storage device, or a cache memory. The storage unit 32 can store data used for the operation of the measurement device 30 and data obtained by the operation of the measurement device 30. For example, the storage unit 32 may store the biological information or the like on the medical care subject measured by the measurement unit 33.

The measurement unit 33 includes at least one sensor that measures the biological information on the medical care subject. Examples of the biological information measured by the measurement unit 33 include, and are not limited to, the body temperature, the blood pressure (systolic blood pressure, diastolic blood pressure, and the like), the blood glucose level, the oxygen saturation (SpO₂), the pulse, the respiration rate, the body weight, the fat weight, the lean body weight, the body water content, the intracellular fluid volume, the extracellular fluid volume, the cardiac output, the activity amount, and the basal body temperature.

The communication unit 34 includes at least one interface for transmitting, to the reading device 20, the biological information on the medical care subject. For example, the communication unit 34 may include an interface of any communication method such as near field communication (NFC), Bluetooth, or a wireless LAN.

Server

As illustrated in FIG. 1, the server 40 includes a control unit 41, a storage unit 42, a communication unit 43, an input unit 44, and an output unit 45. Since configurations of the control unit 41, the storage unit 42, the communication unit 43, the input unit 44, and the output unit 45 of the server 40 are the same as those of the control unit 11, the storage unit 12, the communication unit 13, the input unit 14, and the output unit 15 of the tablet terminal 10, the detailed descriptions thereof are omitted.

Operation of Medical care subject Information Management System

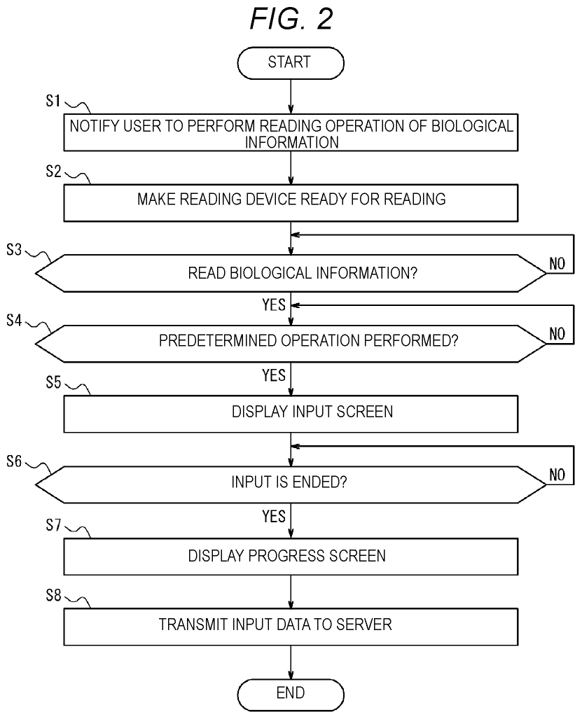

FIG. 2 is a flowchart illustrating an example of the operation of the tablet terminal 10 of the medical care subject information management system. The operation of the tablet terminal 10 described with reference to FIG. 2 may correspond to one of control methods of the medical care subject information management system. The operations executed at steps in FIG. 2 may be executed under control by the control unit 11 of the tablet terminal 10. In the following, as an example, the description will be given of a process which is executed after a physical therapist measures the oxygen saturation of the medical care subject using the measurement device (oxygen saturation measurement instrument) 30 after rehabilitation is performed on the medical care subject.

In S1, the control unit 11 notifies the user (physical therapist) to perform a reading operation of biological information. Specifically, for example, the control unit 11 may cause the display unit of the output unit 15 to display an image for prompting the user to bring the communication unit 34 of the measurement device 30 into contact with a vicinity of the reading unit 23 of the reading device 20.

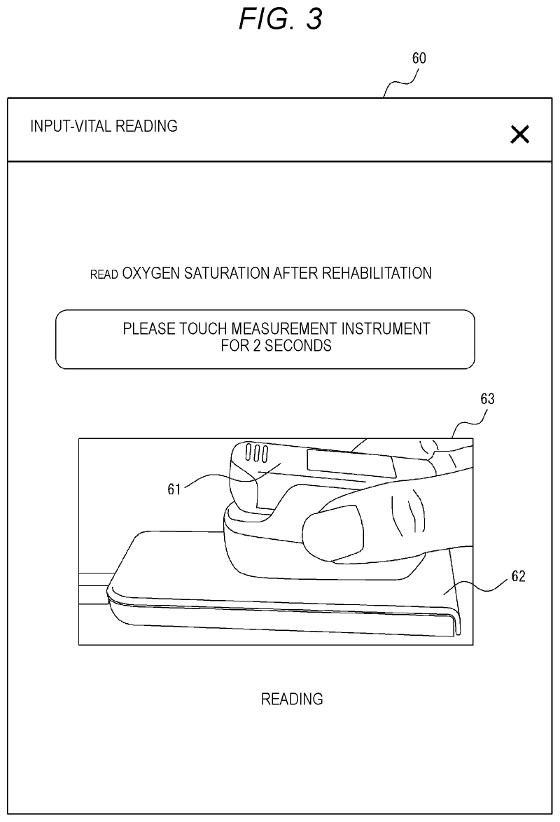

FIG. 3 is a diagram illustrating an example of a screen 60 displayed on the tablet terminal 10 in FIG. 1. In the example in FIG. 3, the screen 60 displays, for example, “please touch measurement instrument for 2 seconds” and displays an image 63 in which the measurement device 30 (“measurement instrument”, 61) is brought into contact with the reading device 20 (62), thereby indicating the reading operation of the biological information (vital sign information) in a relatively easy-to-understand manner.

In S2, the control unit 11 sets the reading device 20 to a state in which the reading device 20 can read the biological information from the measurement device 30. Specifically, the control unit 11 transmits, to the reading device 20 via the communication unit 13, information instructing the reading unit 23 to read the biological information. The control unit 11 may perform a process in S2 before a process in S1.

Upon receiving the instruction from the tablet terminal 10, the reading device 20 controls the reading unit 23 such that the reading unit 23 can read the biological information. In this state, when the user brings the communication unit 34 of the measurement device 30 into contact with the vicinity of the reading unit 23 of the reading device 20, the reading unit 23 of the reading device 20 acquires biological information such as the oxygen saturation of the medical care subject and information such as measurement date and time from the communication unit 34 of the measurement device 30 by NFC communication. When the biological information and the information such as the measurement date and time are acquired from the measurement device 30, the reading device 20 transmits the information to the tablet terminal 10.

In S3, the control unit 11 determines whether the reading device 20 has read the biological information on the medical care subject. Specifically, when the biological information and the information such as the measurement date and time are received from the reading device 20, the control unit 11 determines that the reading device 20 has read the biological information on the medical care subject. If the reading device 20 has read the biological information on the medical care subject (YES in S3), the control unit 11 proceeds to S4, and if not (NO in S3), the control unit 11 continues a process in S3 and waits until the reading device 20 reads the biological information on the medical care subject. The control unit 11 may end the process in the flowchart when the reading of the biological information in the reading device 20 cannot be checked even after a certain period of time (for example, 15 seconds) has elapsed from a state in which the reading device 20 can read the biological information.

In the medical care subject information management system, the read biological information on the medical care subject can be first transmitted from the reading device 20 to the server 40 via the network 50 and then transmitted from the server 40 to the tablet terminal 10 via the network 50. In this case, in response to transmission of the biological information on the medical care subject from the server 40 to the tablet terminal 10, the control unit 11 determines that the reading device 20 has read the biological information on the medical care subject (YES in S3). Further, when the biological information on the medical care subject is transmitted from the reading device 20 to the server 40 via the network 50, not the biological information on the medical care subject but only a fact that the reading device 20 has read the biological information on the medical care subject is transmitted to the tablet terminal 10 as signal information, and the control unit 11 may determine that the reading device 20 has read the biological information on the medical care subject (YES in S3) upon receiving the signal information.

In S4, the control unit 11 determines whether a predetermined operation has been performed by the user. For example, when the biological information and the information such as the measurement date and time are received from the reading device 20, the control unit 11 may display an image (for example, an image such as “next” or “OK”) for proceeding to a next operation together with an image indicating reading completion on the display unit of the output unit 15. When such an image is selected by the user, the control unit 11 may determine that the predetermined operation has been performed. Alternatively, the control unit 11 may determine that the predetermined operation has been performed when a physical button or switch is pressed. If the predetermined operation has been performed (YES in S4), the control unit 11 proceeds to S5, and if not (NO in S4), the control unit 11 continues a process in S4 and waits until the predetermined operation is performed.

The control unit 11 may end the process in the flowchart when the predetermined operation is not performed even after a certain period of time (for example, 15 seconds) has elapsed from the reading of the biological information. Further, the control unit 11 may omit the process in S4. That is, the control unit 11 may immediately proceed to S5 when it is determined in S3 that the biological information has been read.

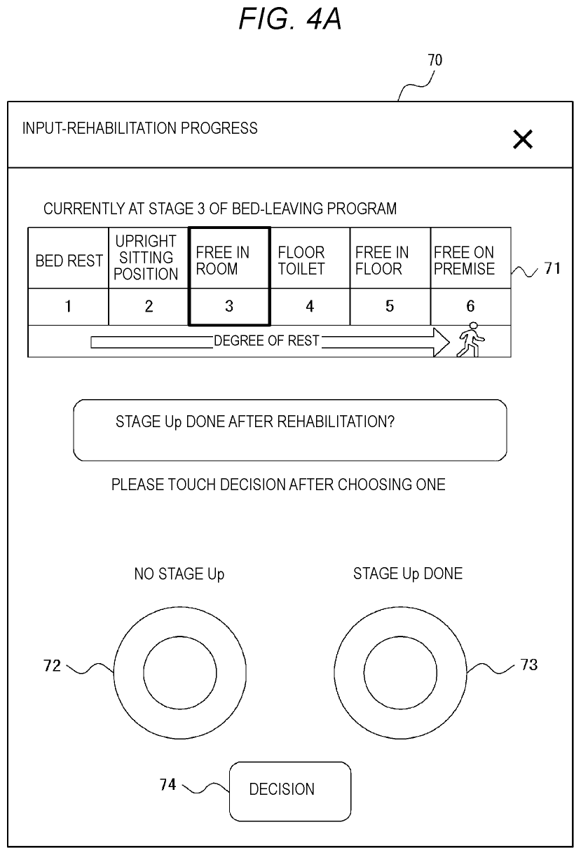

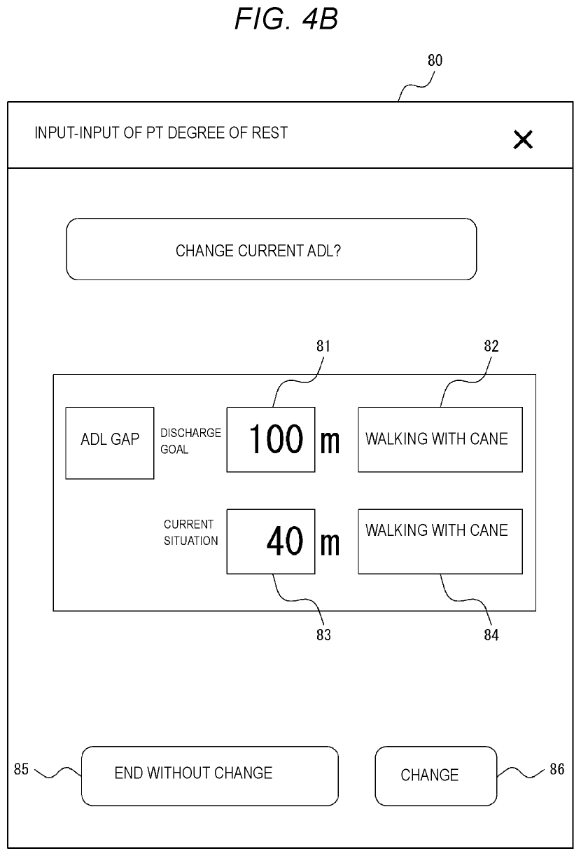

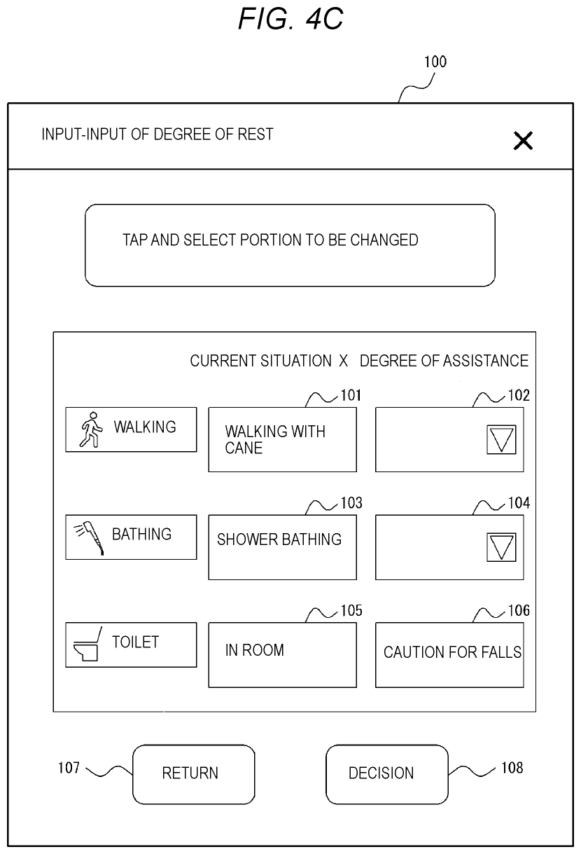

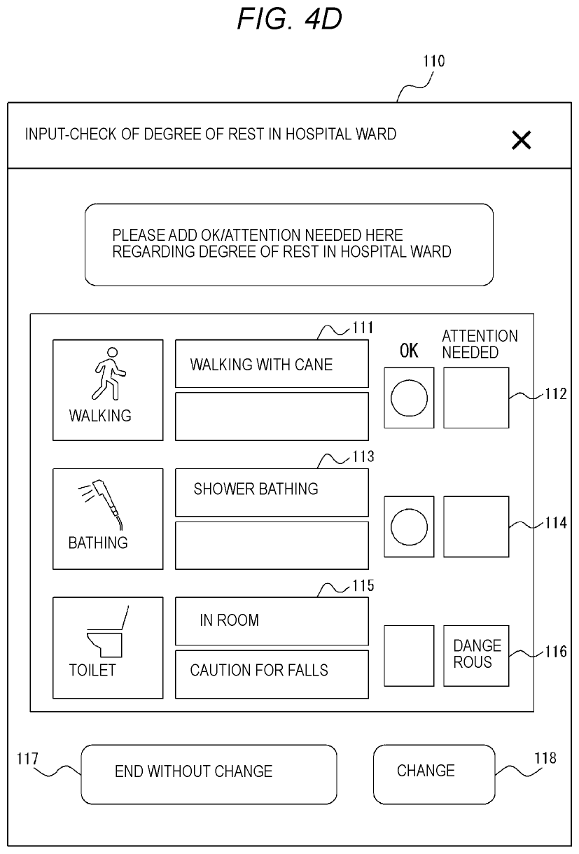

In S5, the control unit 11 causes the display unit of the output unit 15 to display an input screen for inputting information related to the progress of the medical care of the medical care subject. FIG. 4A to FIG. 4D are each a diagram illustrating an example of an image displayed on the tablet terminal 10 in FIG. 1. FIG. 4A to FIG. 4D each illustrate an example of an input screen of the progress information on the medical care subject.

FIG. 4A illustrates an input screen 70 for inputting progress of rehabilitation performed in a bed-leaving program as an example of the input screen of the progress information. The input screen 70 includes an image (progress image) 71 indicating a current progress status of the medical care of the medical care subject. The image 71 indicates the progress status of the rehabilitation, for example, in six stages from a stage 1 “on-bed rest” (it is necessary to rest on a bed) which requires a high need for rest to a stage 6 “free on premise” (it is possible to freely walk around a hospital or other premises) which requires a relatively low need for rest. In the example in FIG. 4A, the image 71 indicates that the current progress status of the rehabilitation of the medical care subject is in a stage 3 “free in room” (it is possible to freely walk in a room such as a hospital room). The input screen 70 may include biological information such as the oxygen saturation read immediately before.

The input screen 70 is displayed to allow the user to select, for example, either an image 72 of “no stage Up” or an image 73 of “stage Up done” as an input of the progress status of the rehabilitation of the medical care subject. When the user determines that a state of the medical care subject after the rehabilitation is recovered to a stage 4 “floor toilet” (it is possible to move to a toilet on the same floor as the hospital room or the like), the user selects the image 73. When the user determines that the state of the medical care subject after the rehabilitation is not recovered from the stage 3, the user selects the image 72. The control unit 11 may further display, on the display unit of the output unit 15 as the input screen 70, an image such as “stage Down done” selected when a symptom of the medical care subject worsens. Further, the control unit 11 may further display, on the display unit of the output unit 15, an image for inputting the stage Up or the stage Down of two or more stages. For example, the control unit 11 may display a plurality of selection images having different numbers of stages for stage Up or stage Down, such as two stages or three stages. Alternatively, for example, the control unit 11 may display images such as “stage Up done” and “stage Down done”, and allow the user to input the number of stages to be shifted based on the number of times of selections of such images. Further, the control unit 11 may accumulate the number of times of selections as +1 when the “stage Up done” is selected and as −1 when the “stage Down done” is selected, and may further display such accumulated values, the number of corresponding stages, and the like as an image on the display unit of the output unit 15. According to such a configuration, the user can rather easily recognize a status of a selection of an image such as the “stage Up done” and the “stage Down done”.

When any one of the images 72 and 73 is selected by a touch operation of the user, the control unit 11 may change a color or the like of the selected image 72 or 73 and display the image 72 or 73 so that the selected image is rather easily distinguished. Further, when any one of the images 72 and 73 is selected by the touch operation of the user, the control unit 11 may change the color or the like of an image 74 of “decision” and display the image 74 so that it is rather easy to distinguish that the image 74 can be selected. After selecting any one of the images 72 and 73, the user selects the image 74 and ends the input operation.

As described above, the tablet terminal 10 displays, as the input screen, a screen on which an option can be selected from among a plurality of options related to the progress of the medical care, instead of displaying the progress status of the medical care subject as a free sentence. Therefore, according to the tablet terminal 10, it is possible to input qualitative information such as the progress of the medical care in a unified format that does not depend on interpretation of a user (an input person and a browsing person). Further, since the user can input the progress of the medical care by a relatively simple selection operation, it is possible to reduce input work burden of the electronic medical record. Further, the tablet terminal 10 displays, as the input screen, a screen (input screen 70) including the image 71 indicating the current progress status of the medical care of the medical care subject on the display unit of the output unit 15. Accordingly, the user can input the progress information while checking the current progress status of the medical care of the medical care subject. Further, when the input screen 70 includes the biological information acquired immediately before, the user can accurately determine presence or absence of the stage Up with reference to the biological information.

FIG. 4B illustrates, as an example of the input screen of the progress information, an input screen 80 for inputting an activity of daily living (ADL) that the medical care subject can perform. In the example in FIG. 4B, the input screen 80 includes input fields for a type of walking (movement method) and a distance that the medical care subject can move by himself/herself by that type of walking. An input field 81 is an area for inputting a distance that the medical care subject can move by himself/herself, which is in a “discharge goal” (goal at discharge). An input field 82 is an area for selecting the type of walking in the “discharge goal”. An input field 83 is an area for inputting a distance that the medical care subject can move by himself/herself in a current situation. The input field 84 is an area for selecting the type of walking in the current situation. The input fields 81 and 82 may display information related to walking in a discharge goal set in advance. The input fields 83 and 84 may display information related to walking in a current situation set in advance.

When the input fields 81 and 83 are selected, the control unit 11 may display a user interface for selecting a numerical value on the display unit of the output unit 15 and receive input of the numerical value. When the input fields 82 and 84 are selected, the control unit 11 may display a pulldown menu in which any one of options such as “independent walking”, “walking with cane”, “walker”, “wheelchair”, “bed leaving”, and “difficulty in bed leaving” can be selected. The user checks information displayed in the input fields 81 to 84, and selects an image 85 of “end without change” when it is not necessary to change the information. When the information displayed in the input fields 81 to 84 is changed, the user selects an image 86 of “change” after inputting the changed information in the input fields 81 to 84.

As described above, the tablet terminal 10 displays, as the input screen on the display unit of the output unit 15, a screen including the input fields 81 and 83 for inputting the quantitative information related to the progress of the medical care of the medical care subject. Further, the tablet terminal 10 displays a plurality of options for the types of walking in the input fields 82 and 84, and receives selections by the user. Therefore, according to the tablet terminal 10, it is possible to input qualitative information such as the progress of medical care in a unified format without depending on subjectivity and interpretation of a user (input person or browsing person) even when various types of users operate the tablet terminal 10. Further, since the input information is input in the unified format, the browsing person can relatively easily and efficiently understand the input information. Further, since the user can input the progress of the medical care by a simple selection operation, it is possible to reduce input work burden of the electronic medical record. Further, the tablet terminal 10 indicates the current progress statuses of the medical care of the medical care subject in the input fields 83 and 84, and receives inputs of changes for the progress statuses. Accordingly, the user can recognize the current progress statuses of the medical care of the medical care subject and efficiently and reliably input information that needs to be changed. Further, the tablet terminal 10 displays, as the input screen, a screen further including, in addition to images (progress images) of the input fields 83 and 84 indicating the current progress statuses of the medical care of the medical care subject, images (goal images) of the input fields 81 and 82 indicating goals (milestones) corresponding to the progress statuses of the medical care of the medical care subject. Accordingly, the user can rather easily recognize a difference (gap) between a goal and a current situation.

Return to the description with reference to FIG. 2. In S6, the control unit 11 determines whether the input of the information related to the progress of the medical care of the medical care subject is ended. If the input of the information is ended (YES in S6), the control unit 11 proceeds to S7, and if not (NO in S6), the control unit 11 continues receiving the input of the information in S6. For example, the control unit 11 may determine that the input of the information is ended when the user selects the image 74 of “decision” in FIG. 4A, the image 85 of “end without change” in FIG. 4B, or the image 86 of “change”.

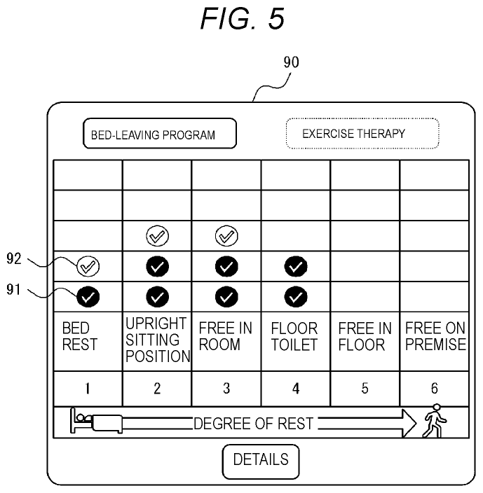

In S7, the control unit 11 displays, on the display unit of the output unit 15, a progress screen showing the progress of the medical care of the medical care subject in detail. FIG. 5 is a diagram illustrating an example of a progress screen 90 displayed on the tablet terminal 10 in FIG. 1. The progress screen 90 shows the progress status of the rehabilitation shown in the image 71 in FIG. 4A in more detail, and may be displayed when, for example, the image 74 of “decision” in FIG. 4A is selected.

The progress screen 90 shows, by patterns 91 and 92, the number of times of rehabilitation performed for the stages (stages 1 to 6) related to the progress of the rehabilitation performed in the bed-leaving program. As illustrated in FIG. 5, every time rehabilitation is performed once in a certain progress stage (for example, the stage 1), the control unit 11 displays the pattern 91 in an order from a lower row of a column corresponding to the stage. As in a case in which the image 73 is selected in FIG. 4A, when the stage of the rehabilitation is ended, the control unit 11 displays the pattern 92 indicating that the rehabilitation at the stage is ended. Accordingly, the user can relatively easily and intuitively recognize, at each of the stages of the progress of the rehabilitation, the number of times of rehabilitation and whether the rehabilitation at this stage is ended.

The rehabilitation is generally performed on a regular basis (for example, daily, every other day, or once a week). In this case, the number of the patterns 91 and 92 displayed in the columns corresponding to the stages of the rehabilitation corresponds to time required for the medical care of the medical care subject at the stages. That is, the progress screen 90 shows, for the stages related to the progress of the medical care, information on the number of times of rehabilitation performed on the medical care subject in the medical care at the stages as time information (information related to time required for the medical care of the medical care subject at the stages). Therefore, the user can rather easily and intuitively recognize how much time is required for the stages of the bed-leaving program based on the number of times of rehabilitation performed at the stages. The control unit 11 may indicate the time information related to the time required for the medical care at the stages related to the progress of the medical care by not only the number of times of rehabilitation but also, for example, the number of days or the number of times of medical examinations. As described above, the tablet terminal 10 displays the medical care progress including the qualitative information in a chart or the like in an easy-to-understand manner. Therefore, the user can instantly view a summary of results, and can rather easily and intuitively grasp the medical care progress status of the medical care subject. Furthermore, the user can compare transitions among the medical care subjects and presume future transitions of the medical care subjects by classifying the medical care progress of the medical care subjects for each tendency.

In S8, the control unit 11 transmits data input by the user to the server 40. The server 40 updates information in the electronic medical record based on the data received from the tablet terminal 10. Then, the control unit 11 ends the process in the flowchart. In addition to or instead of the data input by the user, the control unit 11 may transmit the progress screen 90 to the server 40. Further, the control unit 11 may perform processing in S8 before processing in S7.

As described above, the tablet terminal 10 serving as the medical care subject information management system is communicably connected to the reading device 20 that reads, from the measurement device 30 that measures the biological information on the medical care subject, the biological information measured by the measurement device 30. When the reading device 20 reads the biological information on the medical care subject, the tablet terminal 10 causes the display unit of the output unit 15 to display an input screen for inputting information related to the progress of the medical care of the medical care subject immediately or in response to a predetermined operation. Therefore, since the tablet terminal 10 causes the display unit to display the input screen for inputting the progress information through a series of operations triggered by reading of the biological information using NFC or the like, the user can more efficiently perform reading of the measured biological information and input of the progress information through a continuous operation. Therefore, according to the tablet terminal 10, it is possible to improve handling of information related to a medical care without changing a flow of daily work already established by a health care worker.

In examples in FIG. 3 to FIG. 5, the tablet terminal 10 displays the input screen related to the progress of the medical care related to the bed-leaving program or the ADL in response to the reading of the oxygen saturation. Alternatively, the tablet terminal 10 may display the read biological information such as the oxygen saturation in a graph or the like.

The tablet terminal 10 displays an input screen including an input form in which information related to the progress of the medical care of the medical care subject can be selected from a plurality of options including unified terms or can be input as quantitative information. Therefore, according to the tablet terminal 10, it is possible to input qualitative information such as the progress of the medical care in a unified format that does not depend on interpretation of a user, and it is relatively easy to accurately transmit highly accurate information related to the progress of the medical care. Further, since the tablet terminal 10 receives an input of information related to the progress of the medical care by a selection from options, a numerical value input, or the like, it is possible to reduce input work burden of the user due to narrative descriptions of information.

In response to the input of the information related to the progress of the medical care of the medical care subject via the input screen, the tablet terminal 10 displays, for the stages related to the progress of the medical care, a progress screen showing time information which is information related to the time required for the medical care of the medical care subject at the stages. For example, the tablet terminal device 10 causes the display unit of the output unit 15 to display, as the progress screen for the stages related to the progress of the medical care, a screen showing, as the time information, information on the number of times of rehabilitation performed on the medical care subject in the medical care at the stages. Therefore, the user can rather easily grasp how much time is required for the stages related to the progress of the medical care.

The tablet terminal 10 causes the display unit of the output unit 15 to display an input screen including, as a goal image, an image showing at least one of a most recent achievement goal according to the progress of the medical care of the medical care subject, a final achievement goal, and an achievement goal at the stages related to the progress of the medical care of the medical care subject. Accordingly, the user can recognize a difference between a goal and a current situation when inputting the information related to the progress of the medical care.

In FIG. 4A, the input screen 70 displays a current rehabilitation stage on an upper portion of the screen (the image 71) and displays the images 72 and 73 for inputting presence or absence of a stage up of rehabilitation on a lower portion of the screen, as an example of a case in which the user (input person) is the physical therapist. FIG. 4B illustrates the input screen 80 for displaying a goal and a current situation for an activity of daily living of the medical care subject and allowing the user to input a change.

As another example of the input screen displayed in S5 in FIG. 2, FIG. 4C illustrates, when the input person is a nurse, an input screen 100 for inputting, for each item, a current degree of rest and a current degree of assistance in a rest degree standard used by the nurse. The items of the degree of rest used by the nurse can include, for example, “walking”, “bathing”, and “toilet”, and may also include “changing clothes”, “transfer”, and the like. In the example in FIG. 4C, the input screen 100 includes input fields 101, 103, and 105 for inputting stages of the degrees of rest and input fields 102, 104, and 106 for inputting stages of the degrees of assistance for the items of “walking”, “bathing”, and “toilet”. Each of the input fields 101 to 106 displays the degree of rest and the degree of assistance that are set for the medical care subject, and receives a change in contents from the user. For example, the stages of the degrees of rest for “walking” may include, in a descending order of need for rest, “prohibited”, “only bed leaving”, “inside hospital room”, “inside hospital ward floor”, and “freely inside hospital”. For example, the stages of the degrees of rest for “bathing” may include, in a descending order of need for rest, “wiping on bed”, “shower bathing”, and “bathing”. For example, the stages of the degrees of rest for “toilet” may include, in a descending order of need for rest, “on bed”, “in hospital room”, “near hospital ward”, and “free”. Further, the stages of the degrees of assistance related to the items of “walking”, “bathing”, and “toilet” may include, for example, in a descending order of need for assistance, “assistance needed”, “caution for falls”, “observation needed,” and “none”. In response to a selection of any one of the input fields 101 to 106, the control unit 11 may display options capable of being selected in the input fields 101 to 106 in a drop-down manner and receive a selection from the user. The user checks information displayed in the input fields 101 to 106, and selects an image 107 of “return” when it is not necessary to change the information. When the information displayed in the input fields 101 to 106 is changed, the user selects an image 108 of “decision” after inputting the changed information in the input fields 101 to 106. In response to the selection of the image 108 of “decision”, the control unit 11 registers and updates the information on the medical care subject based on values input to the input fields 101 to 106.

When the user (input person) logs in the tablet terminal 10, the control unit 11 may read the occupation of the user who has logged in from the storage unit 12, and may change display contents, input items, and the like on the input screen displayed on the output unit 15 according to the occupation of the user. For example, when the user who has logged in is a physical therapist, the control unit 11 may display the images 72 and 73 for inputting the presence or absence of the stage Up of the rehabilitation stage on the display unit of the output unit 15 as illustrated in FIG. 4A. Alternatively, when the user who has logged in is a nurse, as illustrated in FIG. 4C, the control unit 11 may display, on the display unit of the output unit 15, current degrees of rest and assistance for “walking”, “toilet”, and “bathing”, and may display the input fields 101 to 106 for inputting changes for the degrees of rest and assistance for “walking”, “toilet”, and “bathing”. As described above, the control unit 11 may cause the display unit of the output unit 15 to display the input screen for inputting the information on the progress of the medical care of the medical care subject, including the display contents according to the occupation of the user who has logged in the medical care subject information management system. Accordingly, since the display contents on the input screen change according to the occupation of the user, the user can efficiently input contents of attention according to the occupation for the same medical care subject.

As another example, for example, when a user of a certain occupation performs an input operation in a situation in which a plurality of input persons input the progress status of the medical care for the same medical care subject, the control unit 11 may change the display contents on the input screen to be provided to a user of another occupation according to input results. For example, a case is considered in which, for a degree of rest and a degree of assistance in a hospital ward, a person of a certain occupation (for example, a physical therapist) serves as an input person (user) and performs an input related to a certain medical care subject via the input screen 100 in FIG. 4C, and a person of another occupation (for example, a nurse) serves as an additional input person and performs an input related to the medical care subject in response to a result of that input. FIG. 4D illustrates an example of an input screen 110 provided to a user of such another occupation.

In FIG. 4D, the input screen 110 includes input fields 111, 113, and 115 set in advance for the items of “walking”, “toilet”, and “bathing” with input results of the degree of rest and the degree of assistance that are input via the input screen 100 in FIG. 4C as initial values. In response to a selection of any one of the input fields 111, 113, and 115, the control unit 11 may display options capable of being selected in the input fields 111, 113, and 115 in a drop-down manner and receive a selection from the user. Input fields 112, 114, and 116 are areas for inputting additional information of “OK” or “attention needed” in response to contents input to the items of “walking”, “bathing”, and “toilet”. The user checks information displayed in the input fields 111 to 116, and selects an image 117 of “end without change” when it is not necessary to change or add the information. When the user adds or changes the information to the input fields 111 to 116, the user selects an image 118 of “change”. In response to a selection of the image 118 of “change”, the control unit 11 registers the changed information on the medical care subject based on values input to the input fields 111 to 116.

For example, the physical therapist performs an input via the input screen 100 in FIG. 4C based on a state of the medical care subject in a rehabilitation room, a result of the medical care, and the like. Thereafter, the nurse additionally inputs information of “OK” or “attention needed” via the input fields 112, 114, and 116 on the input screen 110 in FIG. 4D based on the state of the medical care subject in the hospital ward, the result of the medical care, and the like. The degree of rest and the degree of assistance of the medical care subject are different depending on, for example, a scene to be checked and a checking frequency. Therefore, the tablet terminal 10 receives, via different input screens, inputs of information related to the progress of the medical care checked in the rehabilitation room by the physical therapist and information related to the progress of the medical care checked in the hospital ward by the nurse. The tablet terminal 10 records, as integrated information, information input via an input screen including different display contents depending on occupations and input contents of other users. Therefore, according to the medical care subject information management system, it is possible to more accurately grasp and share a state of a medical care subject.

As described above, when a user of a first occupation performs an input operation as an input person, on the input screen of the information related to the progress of the medical care of the medical care subject, the control unit 11 may determine display contents on the input screen for a user of a second occupation based on the input result of the user of the first occupation. The control unit 11 may cause the display unit of the output unit 15 to display an input screen including the determined display contents for the user of the second occupation. As described above, in a situation in which users of a plurality of occupations input information for the same medical care subject, the tablet terminal 10 changes, according to an input result of a user of a certain occupation, display contents on the input screen for a user of another occupation. Therefore, users of a plurality of occupations having different attention points can smoothly perform cooperative work. Accordingly, it is possible to improve handling of information related to a medical care without changing a flow of daily work already established by a health care worker.

In the above example, the tablet terminal 10 receives an input of information via the input screen 100 in FIG. 4C and then receives an input of a change or addition of the information via the input screen 110 in FIG. 4D, and is not limited to such an operation. For example, the tablet terminal 10 may receive an input of information via the input screen 110 in FIG. 4D and then receive a change of information via the input screen 100 in FIG. 4C. Further, instead of providing the input screen 100 in FIG. 4C to a user of a physical therapist and providing the input screen 110 in FIG. 4D to a user of a nurse, the tablet terminal 10 may, for example, provide the input screen 110 to the physical therapist and provide the input screen 100 to the nurse. Further, the input screens illustrated in FIG. 4A to FIG. 4D are examples, and input screens of other formats and contents may be used. Contents and configurations of an input screen according to an occupation, contents and configurations of an input screen according to input contents of a user of a certain occupation provided to another user, and the like may be selected in view of a type and a characteristic of information related to progress of a medical care, a state of a medical care subject, a flow of an existing medical care in a hospital, and the like.

In order to display different input screens for different occupations, when a user (input person) logs in the tablet terminal 10, the tablet terminal 10 may read an occupation of the user who has logged in from the storage unit 12, and change display contents, input items, and the like on the input screen displayed on the output unit 15 according to the occupation of the user. For example, when the user who has logged in is a physical therapist, the tablet terminal 10 may sequentially display the input screens 70, 80, and 100 in FIG. 4A, FIG. 4B, and FIG. 4C after displaying a vital sign reading screen (screen 60) during rehabilitation as illustrated in FIG. 3. Further, for example, when a user who has logged in is a nurse, the tablet terminal 10 may display the vital sign reading screen (screen 60) as illustrated in FIG. 3 in response to an input operation of a daily vital sign measurement work and then display the input screen 110 in FIG. 4D.

The present disclosure is not limited to the above embodiment. For example, a plurality of blocks illustrated in a block diagram may be integrated, or one block may be divided. Instead of executing a plurality of steps described in the flowchart in time series according to the description, the steps may be executed in parallel or in a different order according to the processing capability of the device that executes the steps or as necessary. In addition, modifications can be made without departing from a gist of the present disclosure. In addition, for example, in the above embodiment, the configurations and the operations of the tablet terminal 10 may be distributed to a plurality of computers capable of communicating with each other. Further, for example, an embodiment is also possible in which some functions of the tablet terminal 10 are provided in another device.

The detailed description above describes embodiments of a medical care subject information management system and a medical care subject information management program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical care subject information management system that is communicably connected to a reading device configured to read, from a measurement device configured to measure biological information on a medical care subject, the biological information measured by the measurement device, or that comprises the reading device, the medical care subject information management system comprising:
a control unit configured to cause a display unit to display an input screen for inputting information related to progress of a medical care of the medical care subject immediately or in response to a predetermined operation performed when the reading device reads the biological information on the medical care subject.

2. The medical care subject information management system according to claim 1, wherein the control unit is configured to cause the display unit to display, as the input screen, a screen for inputting quantitative information related to the progress of the medical care of the medical care subject.

3. The medical care subject information management system according to claim 1, wherein the control unit is configured to cause the display unit to display, as the input screen, a screen on which an option can be selected from among a plurality of options related to the progress of the medical care.

4. The medical care subject information management system according to claim 1, wherein the control unit is configured to cause the display unit to display, as the input screen, a screen including a progress image indicating a current progress status of the medical care of the medical care subject.

5. The medical care subject information management system according to claim 1, wherein in response to an input of the information related to the progress of the medical care of the medical care subject via the input screen, the control unit is configured to cause the display unit to display, for a stage related to the progress of the medical care, a progress screen showing time information which is information related to a time required for the medical care of the medical care subject at this stage.

6. The medical care subject information management system according to claim 5, wherein the control unit is configured to cause the display unit to display, as the progress screen for the stage related to the progress of the medical care, a screen showing, as the time information, information on the number of times of rehabilitation performed on the medical care subject in the medical care at this stage.

7. A medical care subject information management system comprising:
a control unit configured to cause a display unit to display an input screen for inputting information related to progress of a medical care of a medical care subject, the input screen including a progress image indicating a current progress status of the medical care of the medical care subject.

8. The medical care subject information management system according to claim 7, wherein the control unit is configured to cause the display unit to display, as the input screen, a screen further including, in addition to the progress image, a goal image which is an image indicating a goal corresponding to a progress status of the medical care of the medical care subject.

9. The medical care subject information management system according to claim 8, wherein the control unit is configured to cause the display unit to display, as the input screen, a screen including, as the goal image, an image indicating at least one of a most recent achievement goal according to the progress of the medical care of the medical care subject, a final achievement goal, and an achievement goal at each stage related to the progress of the medical care.

10. The medical care subject information management system according to claim 7, wherein the control unit is configured to cause the display unit to display the input screen for inputting the information related to the progress of the medical care of the medical care subject, and the input screen includes display contents according to an occupation of a user who has logged in the medical care subject information management system.

11. The medical care subject information management system according to claim 10, wherein when a user of a first occupation performs an input operation as an input person on the input screen for inputting the information related to the progress of the medical care of the medical care subject, the control unit is configured to determine display contents on the input screen for a user of a second occupation based on an input result of the user of the first occupation, and to cause the display unit to display the input screen including the determined display contents for the user of the second occupation.

12. A medical care subject information management program stored on a non-transitory computer-readable medium that causes a computer that is communicably connected to a reading device configured to read, from a measurement device configured to measure biological information on a medical care subject, the biological information measured by the measurement device, or that includes the reading device to execute a process comprising:
causing a display unit to display an input screen for inputting information related to progress of a medical care of the medical care subject immediately or in response to a predetermined operation performed when the reading device reads the biological information on the medical care subject.

13. The medical care subject information management program according to claim 12, further comprising:
causing the display unit to display, as the input screen, a screen for inputting quantitative information related to the progress of the medical care of the medical care subject.

14. The medical care subject information management program according to claim 12, further comprising:
causing the display unit to display, as the input screen, a screen on which an option can be selected from among a plurality of options related to the progress of the medical care.

15. The medical care subject information management program according to claim 12, further comprising:
causing the display unit to display, as the input screen, a screen including a progress image indicating a current progress status of the medical care of the medical care subject.

16. The medical care subject information management program according to claim 12, wherein in response to an input of the information related to the progress of the medical care of the medical care subject via the input screen, further comprising:
causing the display unit to display, for a stage related to the progress of the medical care, a progress screen showing time information which is information related to a time required for the medical care of the medical care subject at this stage.

17. The medical care subject information management program according to claim 16, further comprising:
causing the display unit to display, as the progress screen for the stage related to the progress of the medical care, a screen showing, as the time information, information on the number of times of rehabilitation performed on the medical care subject in the medical care at this stage.

18. The medical care subject information management program according to claim 12, further comprising:
causing the display unit to display an input screen for inputting information related to progress of the medical care of the medical care subject, the input screen including a progress image indicating a current progress status of the medical care of the medical care subject.

19. The medical care subject information management program according to claim 18, further comprising:
causing the display unit to display, as the input screen, a screen further including, in addition to the progress image, a goal image which is an image indicating a goal corresponding to a progress status of the medical care of the medical care subject; and
causing the display unit to display, as the input screen, a screen including, as the goal image, an image indicating at least one of a most recent achievement goal according to the progress of the medical care of the medical care subject, a final achievement goal, and an achievement goal at each stage related to the progress of the medical care.

20. The medical care subject information management program according to claim 18, further comprising:
causing the display unit to display the input screen for inputting the information related to the progress of the medical care of the medical care subject, and the input screen includes display contents according to an occupation of a user who has logged in the medical care subject information management system; and
wherein when a user of a first occupation performs an input operation as an input person on the input screen for inputting the information related to the progress of the medical care of the medical care subject, determining to display contents on the input screen for a user of a second occupation based on an input result of the user of the first occupation, and causing the display unit to display the input screen including the determined display contents for the user of the second occupation.
